(19) United States
(12) Patent Application Publication
(10) Pub. No.: US 2022/0118129 A1
(43) Pub. Date: Apr. 21, 2022

(54) System and Method for Disinfecting and Drying of Personal Items and Clothing Materials

(71) Applicant: Matthew Steven Ramirez, Alvin, TX (US)

(72) Inventor: Matthew Steven Ramirez, Alvin, TX (US)

(21) Appl. No.: 17/072,532

(22) Filed: Oct. 16, 2020

(51) Int. Cl. A61L 2/10; D06F 58/30; D06F 60/00; D06F 58/38; A61L 2/24

(52) U.S. Cl. CPC A61L 2/10; D06F 58/30; D06F 58/04; D06F 58/38; A61L 2/24; D06F 60/00

(57) ABSTRACT

An ultraviolet (UV) dryer comprising a housing, a first set of UV lights, a control interface, a device memory, and a device processor storing a device application. The housing can comprise a first chamber mounted on said housing, said first chamber comprising a first access door that is configured to enclose said first set of UV lights within the inner surface of said first chamber. The first set of UV lights comprising a first plurality of UV lights. The control interface can be placed at the front panel of said housing, said control interface comprising one or more inputs capable of operating said UV dryer. The device processor can receive input data through said one or more inputs and implement a set of instructions from said input data, wherein one of said set of instructions include disinfecting the daily used items using said first set of ultraviolet (UV) lights.

### BACKGROUND

This disclosure relates to a system and method for disinfecting and drying of personal items and clothing materials. Recently, the need for proper disinfection, sterilization, and drying of daily used items and clothing materials has increased. Traditionally, disinfecting of personal items such as wallets, keys, money, and mobile gadgets are done through washing with the use of antibacterial soap and application of disinfecting liquids or disinfecting sprays. Although the traditional method can be effective in disinfecting items and surfaces, such method can be time consuming and inconvenient especially when sterilization should be done more than once in a day. Moreover, exposure to chemical used for disinfecting liquids, sprays, and cleaning materials can be hazardous to a person's health. Also, washing or applying liquid disinfectant to items such as money, utility bills, and receipts can be deemed impossible as it can cause these items to tear. Another everyday objects that need thorough sterilization can be our clothing. Thus, wash and dry machine can be used to sanitize clothing and linens, however even with washing these fabric materials some microorganism can still remain. Furthermore, commonly-used household appliances are constantly being developed as technology advances. However, operations of dryer and sanitizing machines remain the same. Being able to operate dryer and sanitizing machines remotely can prove beneficial and convenient to users. Additionally, remote operation for sanitizing machine can lessen the exposure of the user from harmful chemicals, UV rays, and microorganisms. As such, it would be useful to have an improved system and method for disinfecting and drying of personal items and clothing materials.

### SUMMARY

An ultraviolet (UV) dryer comprising a housing, a first set of UV lights, a control interface, a device memory, and a device processor storing a device application. The housing can comprise a first chamber mounted on said housing, said first chamber comprising a first access door that is configured to enclose said first set of UV lights within the inner surface of said first chamber. The first set of UV lights comprising a first plurality of UV lights. The control interface can be placed at the front panel of said housing, said control interface comprising one or more inputs capable of operating said UV dryer. The device processor can receive input data through said one or more inputs and implement a set of instructions from said input data, wherein one of said set of instructions include disinfecting the daily used items using said first set of ultraviolet (UV) lights.

A method for disinfecting daily used items using an ultraviolet (UV) dryer is also disclosed. The method can comprise the step of receiving on said UV dryer a set of instructions from one or more inputs. The UV dryer can have a first set of UV lights, the first set of UV lights comprising a first plurality of UV lights, a first chamber comprising a first access door, the first chamber and the first access door configured to enclose the first set of UV lights within the inner surface of said first chamber, a control interface placed at the front panel of said first chamber, said control interface comprising one or more inputs capable of manually operating the UV dryer. The dryer can also have a device memory storing a device application, and a device processor. The method can also comprise the step of implementing the set of instructions received by the UV dryer, wherein one of the sets of instructions can include disinfecting the daily used items through the one or more sets of ultraviolet (UV) lights.

### BRIEF DESCRIPTION OF THE DRAWINGS

FIG. 1 illustrates an ultraviolet (UV) dryer and one or more electronic mobile devices in communication over network.

FIG. 2 illustrates internal components of UV dryer.

FIG. 3A illustrates an embodiment of UV dryer comprising a housing, one or more chambers, one or more sets of UV lights, and a control interface.

FIG. 3B illustrates an embodiment of a set of UV lights using a series circuit.

FIG. 3C illustrates an embodiment of a set of UV lights using a parallel circuit.

FIG. 3D illustrates a front view embodiment of UV dryer.

FIG. 3E illustrates a back-view embodiment of UV dryer.

FIG. 4A illustrates example embodiments of control interface on a UV dryer.

FIG. 4B illustrates an embodiment of control interface displayed on the screen of electronic mobile device.

FIG. 5 illustrates an embodiment of UV dryer comprising a first chamber.

FIG. 6 illustrates another embodiment of UV dryer comprising a second chamber.

FIG. 7 illustrates an internal view of a housing comprising a second chamber.

FIG. 8 illustrates an internal view embodiment of slip ring assembly.

FIG. 9 illustrates a section view embodiment of a second chamber.

FIG. 10 illustrates an electrical circuit diagram of a UV lighting system for a UV dryer.

FIG. 11 illustrates an electrical circuit diagram of a UV dryer.

FIG. 12 illustrates an exemplary method of operating UV dryer.

### DETAILED DESCRIPTION

Described herein is a system and method for disinfecting and drying of personal items and clothing materials. The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure. Accordingly, the claims appended hereto are not intended to be limited by the disclosed embodiments, but are to be accorded their widest scope consistent with the principles and features disclosed herein.

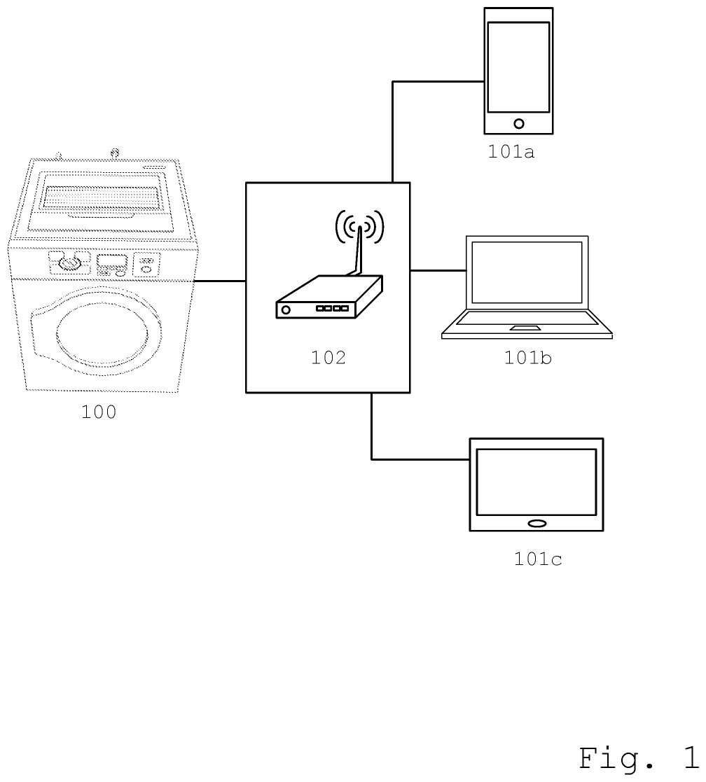

FIG. 1 illustrates an ultraviolet (UV) dryer 100 and one or more electronic mobile devices 101 in communication over network 102. UV dryer 100 can be used for disinfection and sterilization of daily used items, such as clothes, masks, bottles, etc. while drying these items at the same time. In one embodiment, UV dryer 100 can be controlled manually. In such embodiment, a user needs to manually operate UV dryer 100 by actuating the control interface of the dryer. In another embodiment, UV dryer 100 can be controlled remotely using electronic mobile devices 101, which can be further discussed below. Electronic mobile devices 101, can include but is not limited to, a mobile phone, a laptop, a personal digital assistant (PDA), and tablet. Network 102 can be a local area network (LAN), a wide area network (WAN), a piconet, or a combination of LANs, WANs, or piconets. One illustrative LAN is a network within a single business. One illustrative WAN is the Internet. In the preferred embodiment, network 102 will comprise the Internet.

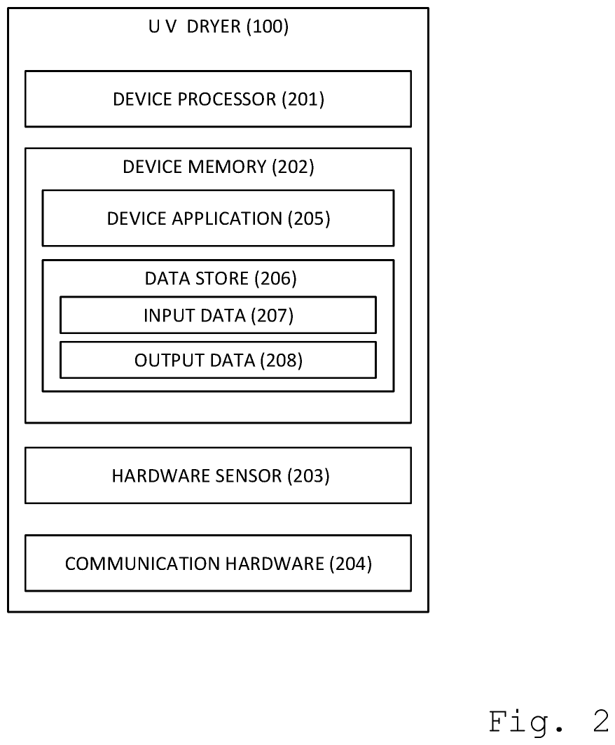

FIG. 2 illustrates internal components of UV dryer 100 comprising a device processor 201, a device memory 202, a hardware sensor 203, and a communication hardware 204. Device processor 201 can be a processing unit that performs a set of instructions stored within device memory 202. Device memory 202 can comprise a device application 205 and data store 206. Device application 205 can comprise business logic for UV dryer 100. In one embodiment, device application 205 can mean local application wherein interface, presentation, logic, and data storage are controlled locally on UV dryer 100. In such embodiment, device application 205 can allow user to control a UV lighting system of UV dryer 100 through the dryer's interface. In such embodiment device memory 205 can mean local memory, device processor 201 can mean local processor, and data store 206 can mean local data. Data store 206 can be collections of data accessible through device application 205. Data store 206 can comprise an input data 207, and an output data 208. In an embodiment wherein UV dryer 100 can be controlled manually, input data 207 can be sent to UV dryer 100 when inputs on UV dryer 100 are actuated. In another embodiment wherein UV dryer 100 can be controlled remotely, input data 207 can be any information or set of instructions sent to UV dryer 100 from electronic devices 101 via network 102. Output data 208 can be set of instructions performed by UV dryer 100 in response to input data 207. Hardware sensor 203 can include but is not limited to temperature sensor and moisture sensor that is capable of monitoring the conditions of UV dryer 100 to achieve optimal dry for each load. In one embodiment, input data 207 can be collected from hardware sensor 203 and can be sent to device processor 201.

Communication hardware 204 can include a network transport processor for packetizing data, communication ports for wired communication, or an antenna for wireless communication. Further, input data 207 and output data 208 can be sent to communication hardware 204 for communication over network 102. In one embodiment, communication hardware 204 can allow transmitting and receiving communication with electronic mobile devices 101 via Bluetooth. In a preferred embodiment, communication hardware 204 can allow UV dryer 100 to transmit and/or receive data from electronic mobile devices 101 through a wireless network connection.

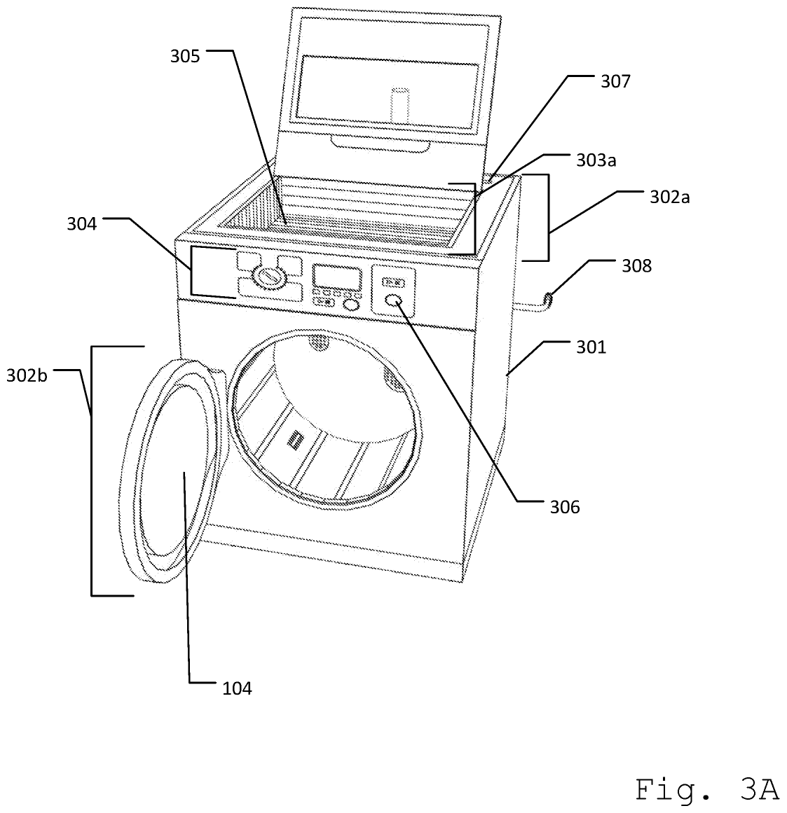

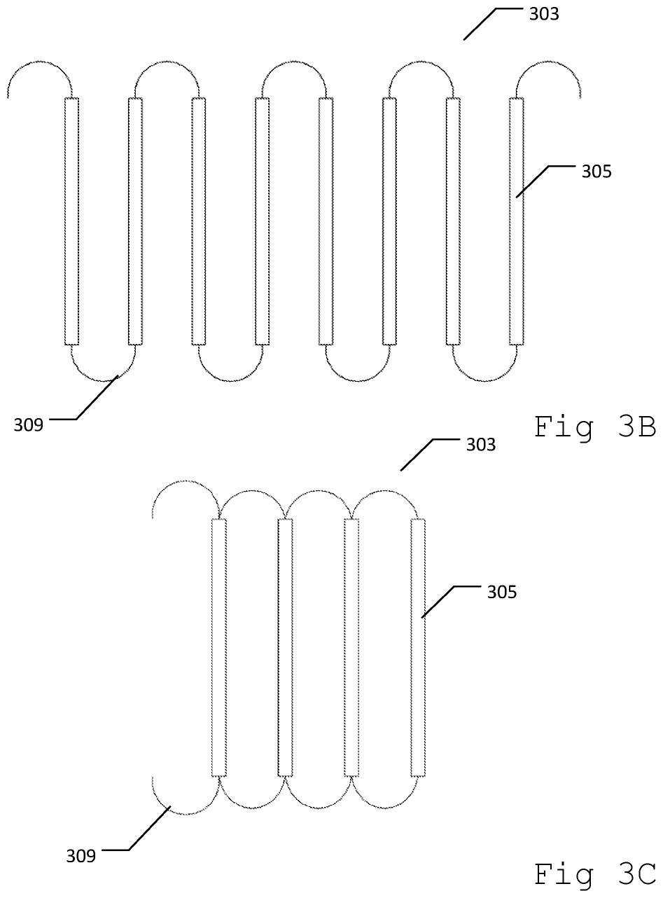

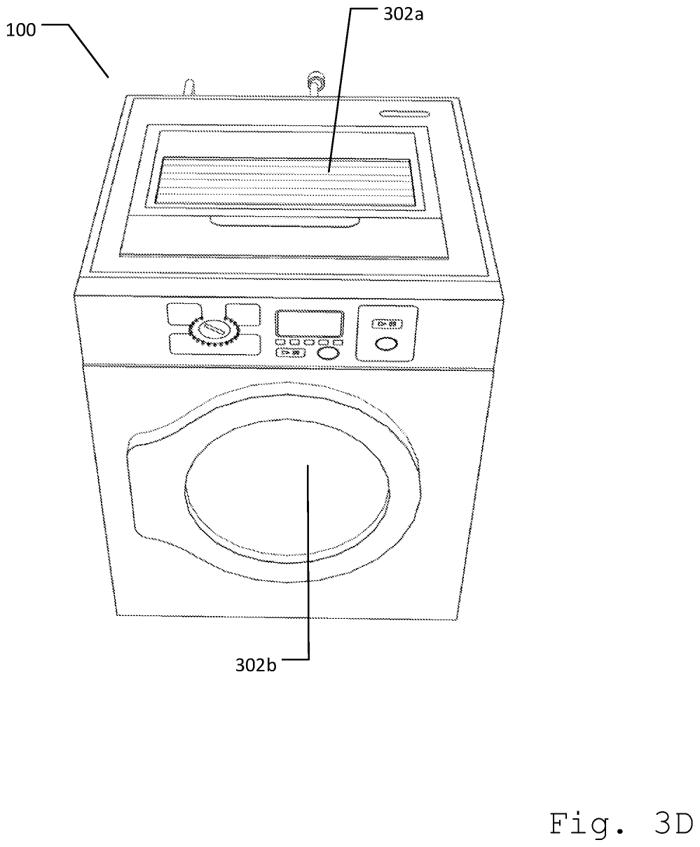

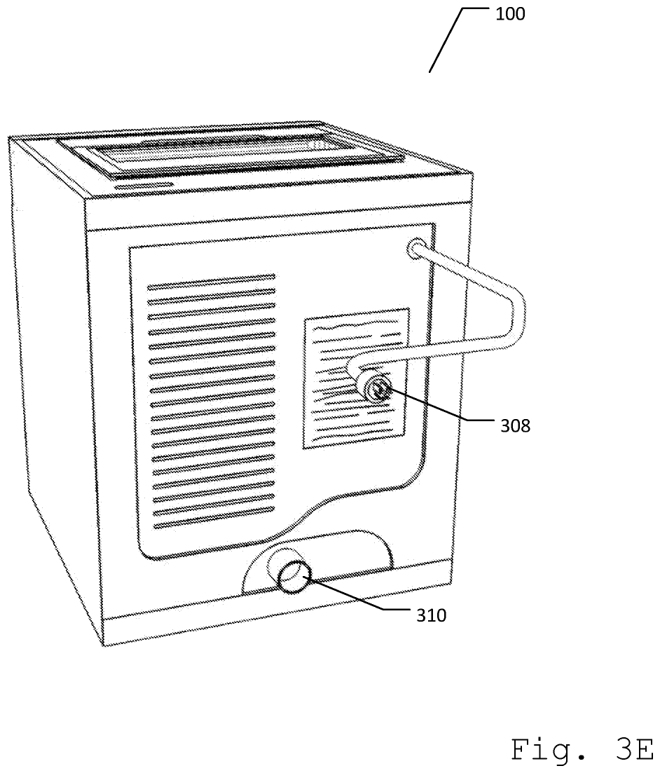

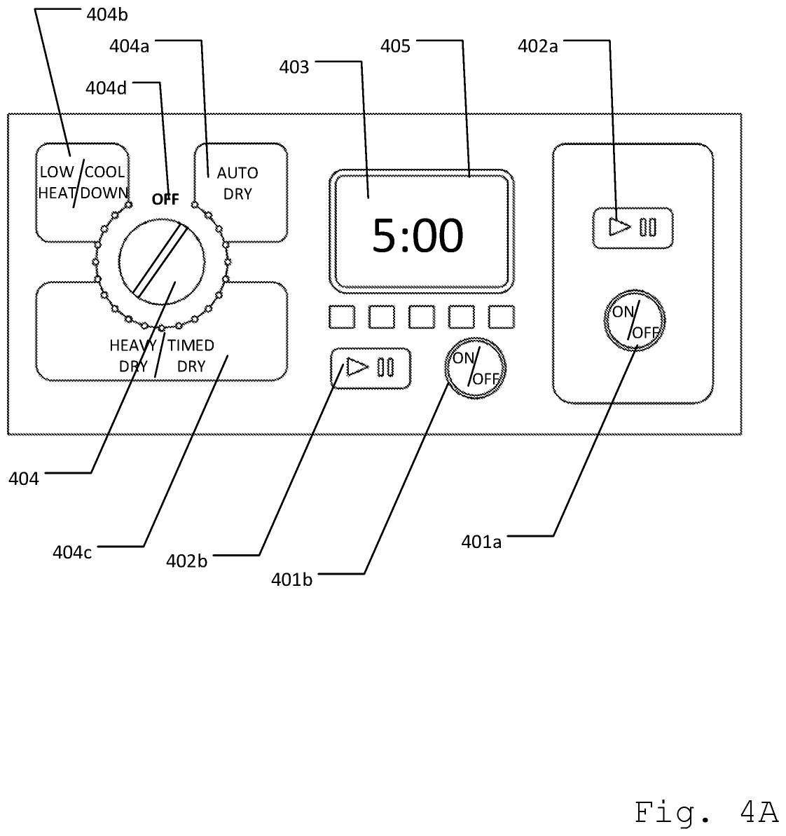

FIG. 3A illustrates an embodiment of UV dryer 100. In one embodiment, UV dryer 100 can comprise a housing 301, one or more chambers 302, one or more sets of UV lights 303, and a control interface 304. Housing 301 can be an enclosed container capable of mounting chambers 302. Chambers 302 can be an enclosure capable of receiving daily used items, and fabric materials that needs drying and sterilization. In one embodiment, each chamber 302 can comprise one set of UV lights 303. Each set of UV lights 303 can comprise a plurality of UV lights 305. UV lights 305 can disinfect surfaces since the ionizing effects of UV radiation can kill microorganisms. When bacteria, viruses and protozoa are exposed to the germicidal wavelengths of UV light, these microorganisms become incapable of reproducing and infecting individuals. In such embodiment, chamber 302 can be capable of drying and/or sterilizing items mounted within the chamber enclosure. In one embodiment, UV dryer 100 can comprise a first chamber 302a. In one embodiment, first chamber 302a can comprise a first set of UV lights 303a configured to disinfect sterilize personal items and different objects, which can include but are not limited to masks, gloves, pacifiers, wallets, keys, tableware, utensils, and baby bottles. In another embodiment, UV dryer 100 can comprise a second chamber 302b. Second chamber 302b can be capable of cool-drying or heat-drying fabric materials such as clothes and linens. In one embodiment, second chamber 302b can further comprise a second set of UV lights 303b. In such embodiment, second chamber 302b can be configured to dry and disinfect fabric materials placed within the second chamber. In a preferred embodiment, UV dryer 100 can comprise both first chamber 302a and second chamber 302b. In this embodiment, first chamber 302a can be placed at the top portion of housing 301, while second chamber 302b can be placed below first chamber 302a. Control interface 304 can be capable of controlling the operations of UV dryer 100. In one embodiment, control interface 304 can be placed at the front surface of housing 301. In one embodiment, control interface 304 can comprise a plurality of inputs 306. For purposes of this disclosure, control interface 304 can comprise of input device and output device. Input devices can be any piece of equipment that provides data and control signals of UV dryer 100. In one embodiment, input devices on UV dryer 100 can comprise inputs 306 and sensors. Inputs 306 can be configured to send input data 207 to device processor 201. Some examples of inputs 306 can include but are not limited to buttons, switches, dials, timers, and meters for controlling the drying and disinfecting routines of UV dryer 100. In another embodiment, input devices can comprise a plurality of keys (or keyboard) of electronic mobile device 101 capable of sending input data 207 to device processor 201. Further, output devices can be any piece of equipment that can convert input data 207 received from input devices into human-readable form. Some examples of output devices can include but are not limited to light emitting diode (LED) indicators, a speaker, and a screen. In one embodiment, output devices can display data through the screen on control interface 304 of UV dryer 100. In another embodiment, UV dryer 100 can be capable of transmitting output data 208 and display output data 208 on the screen of electronic mobile devices 101.

Additionally, UV dryer 100 can further comprise a lint filter 307, and a power plug 308. Lint filter 307 can catch lint, or any dirt particles released from fabric materials while drying and sterilizing it using UV dryer 100. Power plug 308 can connect UV dryer 100 to a power supply, which can provide electrical power needed to operate UV dryer 100.

FIG. 3B illustrates an embodiment of a set of UV lights 303 using a series circuit. In this embodiment, each set of UV lights 303 can comprise UV lights 305. In such embodiment, each UV light 305 can be connected in a series circuit through a pair of wires 309. In such embodiment, the current that flows through each set of UV lights 303 can only have one path.

FIG. 3C illustrates another embodiment of a set of UV lights 303 using a parallel circuit. In this embodiment, each set of UV lights 303 can be connected in a parallel circuit. In such embodiment, each UV light 305 can be connected across each other through wires 309, and can form two sets of electrical common points. Further in another embodiment, one set of UV lights 303 can be connected in a series circuit while the other set of UV lights 303 can be connected in a parallel circuit.

FIG. 3D illustrates a front view embodiment of UV dryer 100.

FIG. 3E illustrates a back-view embodiment of UV dryer 100. In one embodiment, the back surface of UV dryer 100 can comprise power plug 308, an exhaust vent 310.

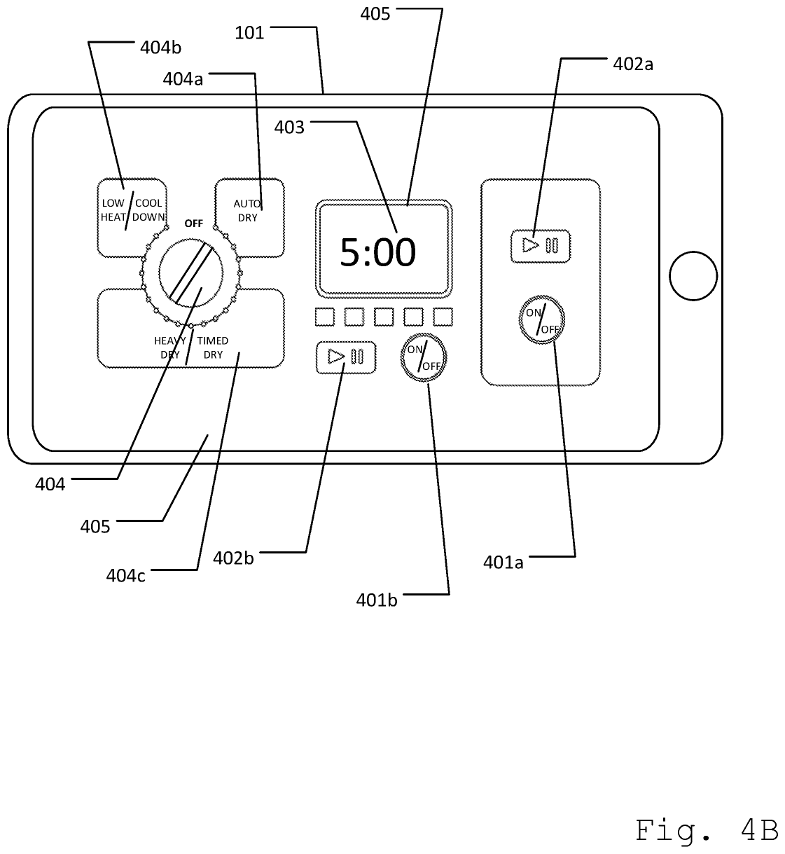

FIG. 4A illustrates an example embodiment of control interface 304 on UV dryer 100. In this embodiment, UV dryer 100 can be operated manually by interfacing with the inputs on control interface 304. In one embodiment, control interface 304 can control operations of each chamber 302. In an embodiment wherein UV dryer 100 comprises first chamber 302a and second chamber 302b, control interface 304 can be capable of controlling first chamber 302a and second chamber 302b independently. In such embodiment, a user can use first chamber 302a separately such that the user can choose to sterilize personal items on first chamber 302a while second chamber 302b can be turned off, and vice versa.

As a non-limiting embodiment, control interface 304 can comprise of inputs that can include but are not limited to an on-off button 401, a start-stop button 402, a timer 403, and a function dial 404. In one embodiment, first chamber 302a can be controlled using a top-dryer on-off button 401a, and a top-dryer start-stop button 402a. In such embodiment, actuating top-dryer buttons 401a and 402a can control the operations of first chamber 302a. Top-dryer on-off button 401a can turn on first chamber 302a, while top-dryer start-stop button 402a can start and stop the drying function and sterilization of first chamber 302a. As such, actuating top-dryer start-stop button 401a can control the UV lights on first chamber 302a to turn on or off. Timer 403 can be displayed through a screen 405, which shows the length of time the objects within first chamber 302a are dried and sterilized.

In another non-limiting embodiment, second chamber 302b can be controlled using inputs that can include but are not limited to a bottom-dryer on-off button 401b, and a bottom-dryer start-stop button 402b. Actuating bottom-dryer on-off button 401b can allow second chamber 302b to turn on or off while a bottom dryer start-stop button 402b can turn on or turn off the UV lights on second chamber 302b. Function dial 404 can be used to operate the different dryer functions of second chamber 302b. Some example of dryer functions can include but are not limited to an auto-dry function 404a, a cool-dry function 404b, heat-dry function 404c, and off function 404d. Heat-dry function 404c can be used when drying damp fabric materials while selecting cool-dry function 404b can be used to disinfect dry fabric materials. Further in one embodiment, selecting auto-dry function 404a can allow the sensors in UV dryer 100 to operate. In such embodiment, UV dryer 100 can suggest which settings or function will best suit the items detected within second chamber 302b. In this embodiment, the sensors after detecting the items placed within a rotating drum of second chamber 302b can send input data 207 to device processor 201, the device processor can then analyze the input data and then send a set of instructions to a screen 405. Screen 405 can then display a suggested settings or functions to be used. Moreover, screen 405 can display output data 208 received from the sensors within second chamber 302b. Thus, screen 405 can also display humidity information such as moisture and air temperature within second chamber 302b. In some embodiment, control interface 304 can also comprise a speaker and LED (light-emitting diode) indicators. In such embodiment, the speaker can play a sound and cause LED indicators to blink whenever the operation of first chamber 302a and/or the operation of second chamber 302b has ended. In some embodiments, screen 405 can also display the remaining time left on the operation of UV dryer 100.

FIG. 4B illustrates an embodiment of control interface 304 displayed on the screen of electronic mobile device 101. In this embodiment, a user can also control and monitor the functions of UV dryer 100 remotely by using electronic mobile device 101 connected through network 102. In one embodiment, device application 205 displayed on electronic mobile device 101 can look similar with the physical inputs on control interface 304. In this embodiment, controls and functions of UV dryer 100 can be accessed and manipulated remotely using electronic mobile device 101.

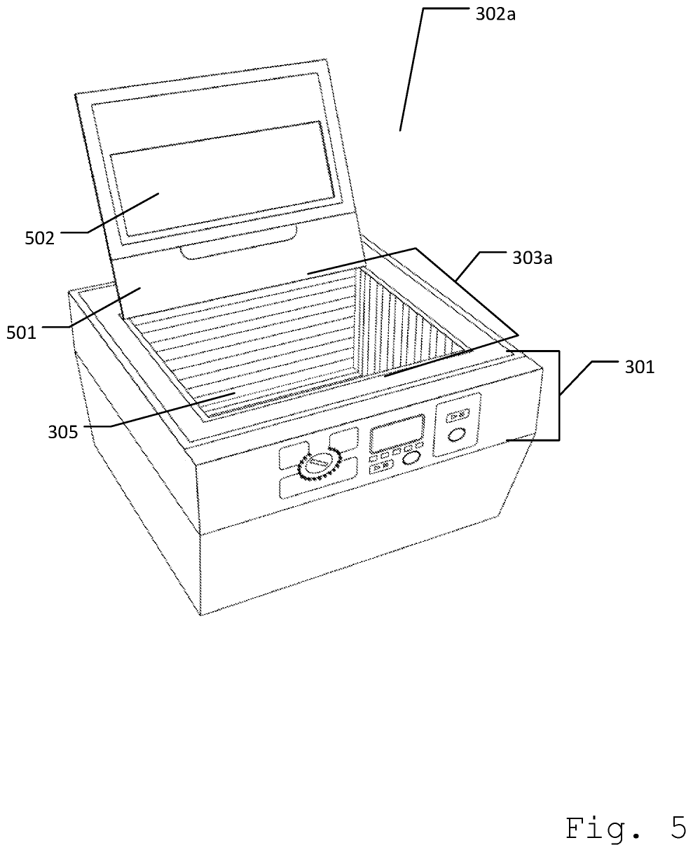

FIG. 5 illustrates an embodiment of UV dryer 100 comprising first chamber 302a. In this embodiment, first chamber 302a can comprise first set of UV lights 303a and a first access door 501. In one embodiment, first set of UV lights 303a can be placed around the inner surface of first chamber 302a. In one embodiment, first chamber 302a can be a deep enclosure capable of mounting daily used items such as wallets, paper bills, keys, eyewear, gloves, hats, masks, and small kitchen wares. In such embodiments, first chamber 302a can be used to dry and sterilize daily used items. Furthermore, it can only take anywhere from 12.5 to 27.5 minutes to disinfect and sterilize items within first chamber 302a depending on the quartz glass used for the UV lights. First access door 501 can ensure that items placed within first chamber 302a are enclosed within the chamber. In one embodiment, first chamber 302a cannot be actuated if first access door 501 is not securely closed. Further in one embodiment, first access door 501 can comprise a UV filter 502 that is configured to block UV rays from passing through first access door 501.

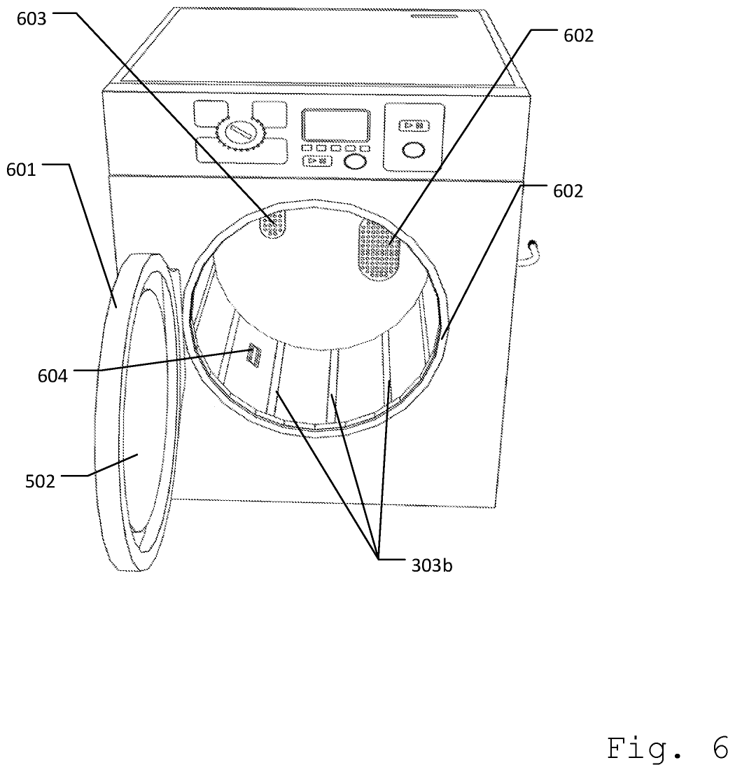

FIG. 6 illustrates another embodiment of UV dryer 100 comprising second chamber 302b. In this embodiment, UV dryer 100 can comprise a second access door 601. Second access door 601 can allow a user to access inner surface of second chamber 302b. In one embodiment, second access door 601 can comprise UV filter 502 that can block ultraviolet rays from passing through. This can prevent the user from being exposed to the radiation of UV lights when using UV dryer 100. In this embodiment, second chamber 302b can be can be a cylindrical tube configured to rotate around a fixed axis of housing 301. In one embodiment, second chamber 302b can comprise second set of UV lights 303b, a dryer blower 602, a cool air dryer 603, and a humidity sensor 604. In one embodiment, each UV light 305 on second set of UV lights 303b can be spatially placed around the middle layer of second chamber 302b such that each UV light 305 can be parallel to the central axis of second chamber 302b. Dryer blower 602 and cool air dryer 603 can be placed at the back-end surface of second chamber 302b. In an embodiment wherein function dial 404 can be selected on heat-dry function 404c, dryer blower 602 can heat-dry fabric materials within second chamber 302b. In another embodiment wherein cool-dry function 404b is selected, cool air dryer 603 can cool-dry (air dry) fabric materials within second chamber 302b. Further in another embodiment actuating bottom-dryer start-stop button 402b, can turn on or off the UV lights within second chamber 302b. In an embodiment wherein function dial 404 can be set to off 404d and bottom-dryer start-stop button 402b is actuated, second chamber 302b can start to operate and rotate without the functions of both dryer blower 602 and cool air dryer 603. In such embodiment, only the UV lights within second chamber 302b can be turned on. As such, fabric materials within second chamber 302b can be tossed within the chamber while UV lights are turned. Thus, second chamber 302b can allow dried fabric materials be sterilized and disinfected within the chamber.

Further, dryer blower 602 can blow heated air that can cause the moisture on fabric materials to evaporate while cool air dryer 603 can blow cool air to fabric materials within second chamber 302b. Humidity sensor 604 can be placed near the front inner surface of second chamber 302b. Humidity sensor 604 can measure both moisture and temperature inside second chamber 302b. In one embodiment, once humidity sensor 604 senses that fabric materials within the chamber is dry enough, humidity sensor 604 can automatically turn off UV dryer 100. In another embodiment, humidity sensor 604, after sensing the dampness of fabric materials within the chamber, can transmit the data to device processor 201. In return, device processor 201 can be capable of selecting a preset dryer setting needed to dry materials within second chamber 302b. In one embodiment, preset dryer setting can be the length of time needed to dry the materials within the second chamber. In another embodiments, preset dryer setting can be a function setting such as cool drying, or heat drying of the materials within the second chamber.

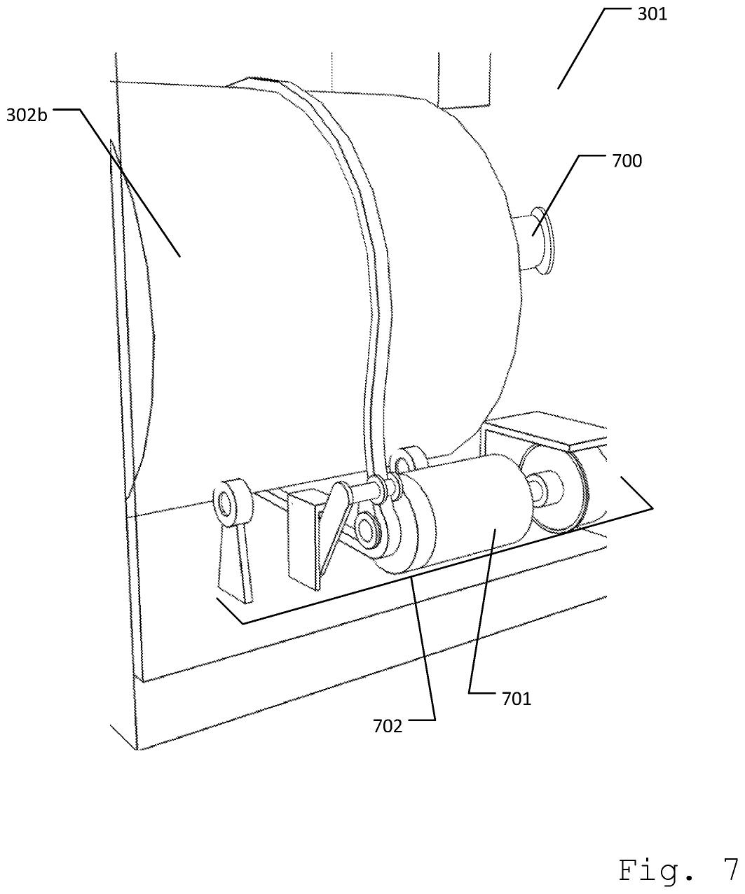

FIG. 7 illustrates an internal view of housing 301 comprising second chamber 302b. In this embodiment, second chamber 302b can be a rotating drum. For purposes of this disclosure, rotating drum can be the component of a dryer machine that is configured to rotate causing fabric materials to toss and tumble within the drum. In one embodiment, second chamber 302b can mount a slip ring assembly 700. Slip ring assembly 700 can be a method of making an electrical connection from a stationary object to a rotating object. In this embodiment, electrical energy can be transferred from stationary portion of UV dryer 100 to the rotating portion of UV dryer 100, which is second chamber 302b. Further, in one embodiment, second chamber 302b can be connected to an electric motor 701 through a roller system 702. Electric motor 701 can transmit electrical energy from the power source to roller system 702. An example of roller system 702 can be a pulley system. Once power is transmitted to roller system 702, the electrical current can cause second chamber 302b to rotate. As second chamber 302b rotates, dryer blower 602 can release heated air within the second chamber. As fabric materials are rotated the moisture from the materials are drained away with the help of heated air from dryer blower 602.

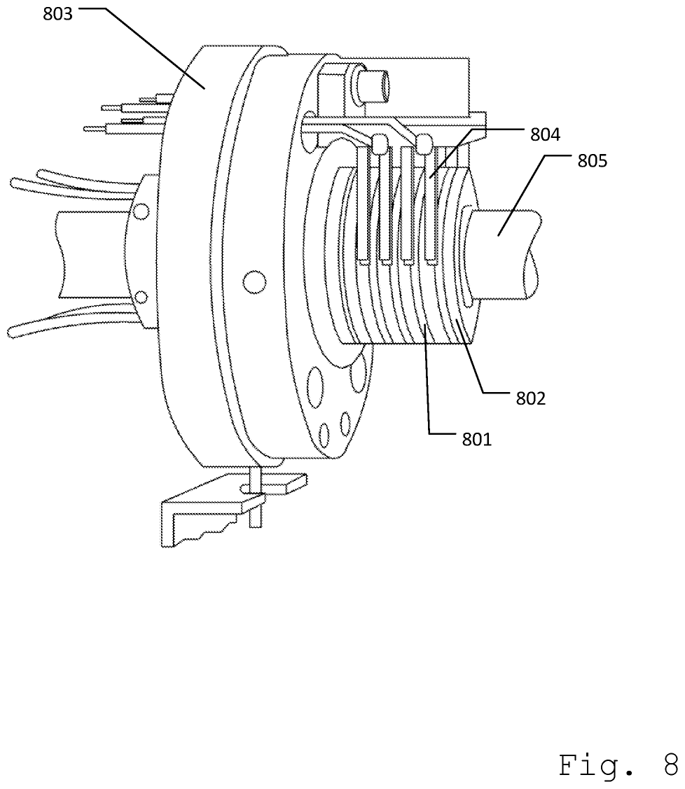

FIG. 8 illustrates an internal view embodiment of slip ring assembly 700. In one embodiment, slip ring assembly 700 can comprise of a plurality of slip rings 801, a rotor 802, a stator 803, a set of brushes 804, and an axle 805. Rotor 802 can be the rotating component of slip ring assembly 700 while stator 803 can be the stationary component. In one embodiment, stator 803 can connect to a portion of housing 301, while rotor 802 can connect to second chamber 302b. In this embodiment, rotor 802 can comprise slip rings 801. Brushes 804 can connect to slip rings 801, which can allow the electrical current or signal be conducted between stator 803 and rotor 82. In this embodiment, axle 805 can connect to rotor 802. In one embodiment, second chamber 302b can mount axle 805.

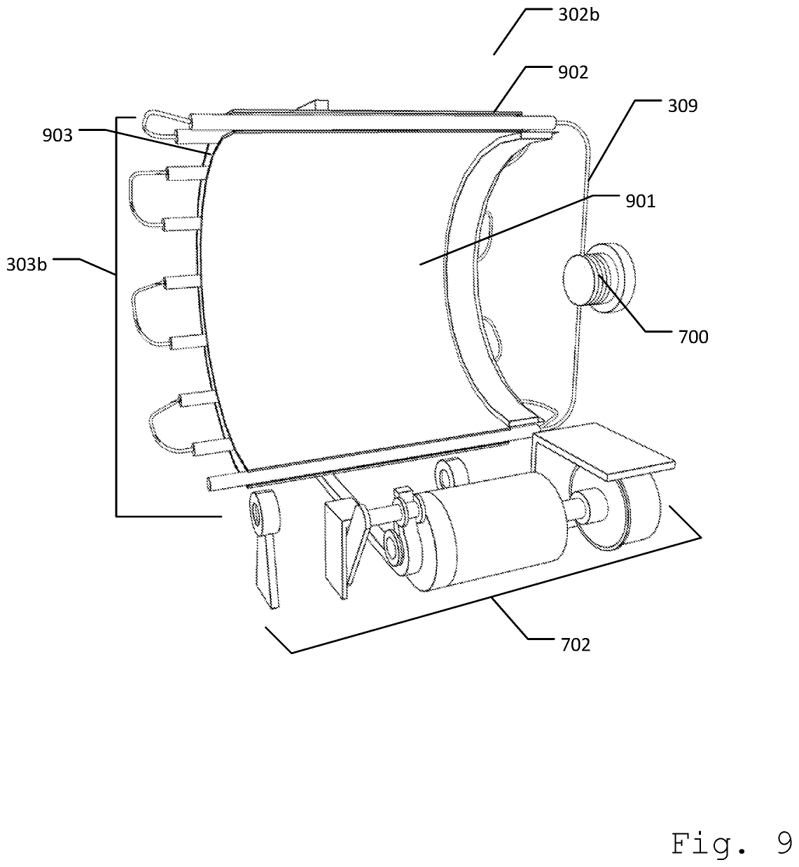

FIG. 9 illustrates a section view embodiment of second chamber 302b. In one embodiment, second chamber 302b can comprise an inner surface 901, an outer surface 902, and a middle layer 903. Inner surface 901 can be the surface of second chamber 302b that comes in contact with fabric materials that needs sterilization and drying. Outer surface 902 can be the outer layer of second chamber 302b that comes in contact with roller system 702. Middle layer 903 can be the portion in between inner surface 901 and outer surface 902. In this embodiment, second set of UV lights 303b can be mounted within middle layer 903. As such, second set of UV lights 303b can be connected to slip ring assembly 700 through wires 309. Axle 805 can mount second chamber 302b such that rotor 802 can rotate with second chamber 302b. In this structure, slip ring assembly 700 can transmit electrical current from stator 803 to rotor 802. In such structure, slip ring assembly 700 can transmit electrical current to power second set of UV lights 303b as second chamber 302b rotates. Thus, as second chamber 302b rotates the drum can allow fabric materials to dry and be sterilized. Further, the air that leaves UV dryer 100 can pass through lint filter 307. The exhausted air can then pass through exhaust vent 310.

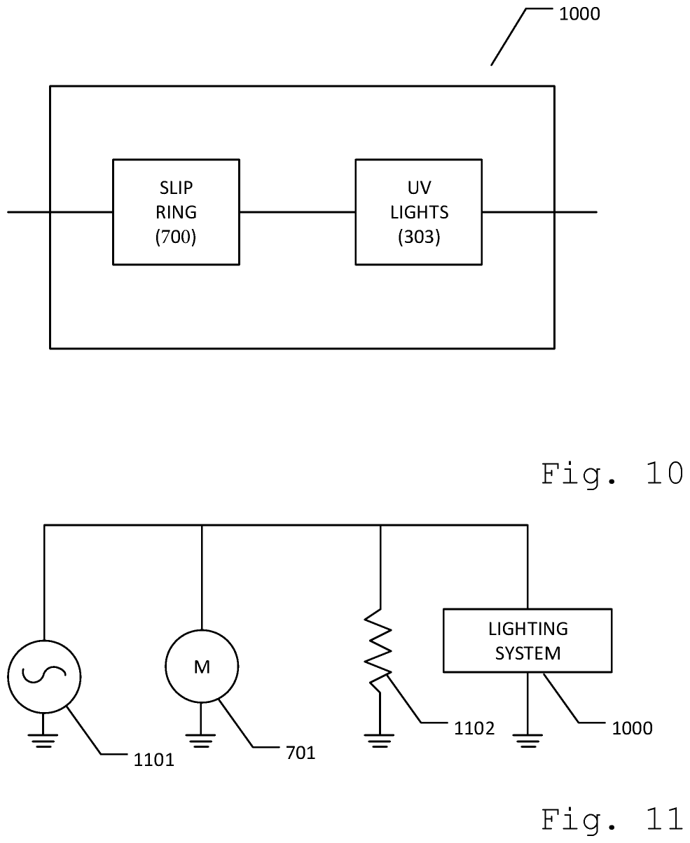

FIG. 10 illustrates an electrical circuit diagram of a UV lighting system 1000 for UV dryer 100. In an embodiment wherein UV dryer 100 can comprise second chamber 302b, second set of UV lights 303b can connect to ring assembly 700 through wires 309. In such structure, electric current that passes through slip ring assembly 700 can be transmitted to second set of UV lights 303b. In such embodiment, when device processor 201 receives a signal from a power source, the signal gets transmitted to slip ring assembly 700 and cause rotor 802 to rotate. Such rotation can allow brushes 804 to rub with slip rings 801 causing the electric current or signal be conducted. As such, as rotation occurs on ring assembly 700, the electric current gets transmitted to second set of UV lights 303b. This can then allow second set of UV lights 303b to operate and produce light within second chamber 302b.

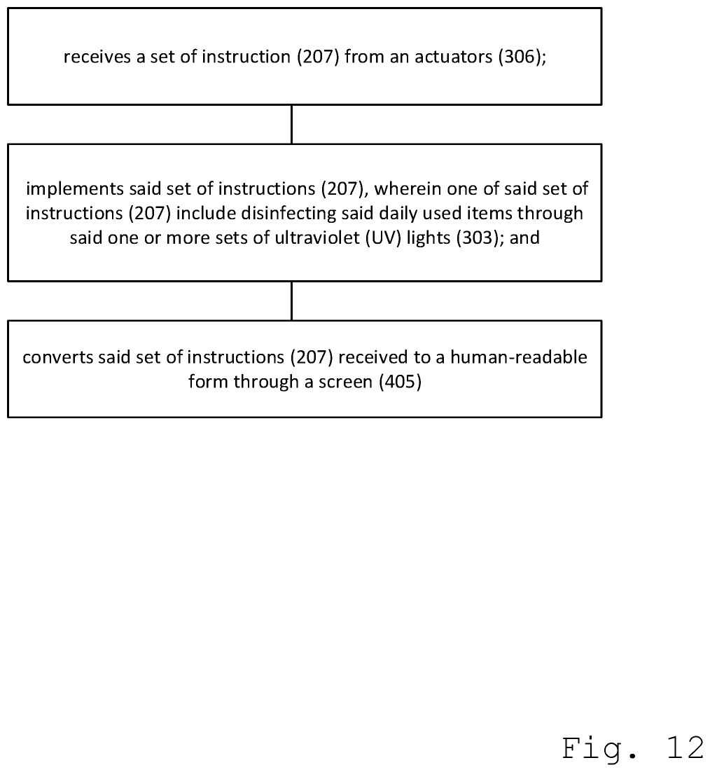

FIG. 11 illustrates an electrical circuit diagram of UV dryer 100. In this embodiment, UV dryer 100 can comprise electric motor 701, UV lighting system 1000, an alternating current (AC) voltage source 1101, and a load 1102. Once power plug 308 of UV dryer 100 is plugged into a power source and UV dryer 100 is turned on, the electric current can flow through electrical circuit of UV dryer 100. After selecting a dryer function on control interface 304, device processor 201 can control the transmission of electric current from AC voltage source 1101 to electric motor 701, load 1102, and lighting system 1000. The current from AC voltage source 1101 can be transmitted to power electric motor 701. The signal received from voltage source 1101 can allow electric motor 701 to operate and can cause objects within the chambers to dry. As such, the electrical signal can also be transmitted to load 1102. In one embodiment, load 1102 can be other components of UV dryer 101 that can consume electric power, such as LED light indicators and speakers. The electrical current can also be transmitted to lighting system 1000 that can power the UV lights within UV dryer 100, as discussed above.

FIG. 12 illustrates an exemplary method of operating UV dryer 100. In an embodiment wherein UV dryer 100 can comprise both first chamber 302a and second chamber 302b, a user can use both chambers at the same time. Initially, the user can load daily used items into first chamber 302a and then load fabric materials into second chamber 302b. Once loaded, the user can start operating UV dryer 100 by interfacing with the dryer inputs 306. In one embodiment, the user can control UV dryer 100 through manually interfacing with inputs 306 on control interface 304. In another embodiment, the user can access and control UV dryer 100 through accessing device application 205 through one of electronic mobile devices 101. After loading the daily items and/or fabric materials into UV dryer 100, the user can then start disinfecting the loaded items. The user can start operating UV dryer 100 by selecting a function to use for drying and disinfecting the items loaded in the chambers. Input data 207 can either be transmitted to UV dryer 100 from control interface 304 of UV dryer 100 or from device application 205 of electronic mobile device 101. Upon receiving input data 207, UV dryer 100 can implement the set of instructions, wherein one of the set of instructions can include disinfecting the items within UV dryer 100. In a scenario wherein first chamber 302a is loaded with daily items, interfacing with top-dryer start-stop button 402a can turn on first set of UV lights 303a within first chamber 302a. As such, first chamber 302a can allow items within the casing to be disinfected and sterilized. In one embodiment, the user can input data 207 such as length of time the items are to be sterilized using inputs 306. Timer 403 can then be displayed on screen 405, which can display the time left for items to dry or time left to complete sterilization of the items. In another scenario, second chamber 302b can be operated after loading the drum with fabric materials. Hardware sensor 203 can then monitor the condition within second chamber 302b, and then transmits the humidity condition within the drum as output data 208. Output data 208 can then be displayed on screen 405 of control interface 304, in one embodiment. In another embodiment, output data 208 can be displayed on device application 205 through the screen of electronic mobile device 101. Furthermore, in such scenario, UV dryer 100 can be configured to preselect an ideal preset dryer setting to dry and disinfect the materials within second chamber 302b. The user can then choose to follow the preset dryer settings. As such, UV dryer 100 can implement the selected preset dryer setting while disinfecting the fabric materials within second chamber 302b. In another scenario, the user can choose to override the preselected dryer setting and select his preferred setting by interfacing with inputs 306 of UV dryer 100. Once selected, UV dryer 100 can implement the preferred dryer settings selected. As second chamber 302b operates within housing 301, slip ring assembly 706 can transmit signals to power second set of UV lights 303b. Thus, allowing fabric materials within second chamber 302b be dried while being disinfected and sterilized at the same time.

Operating system(s) can be stored in device memory 202 and executable by device processor 201. Other applications can be stored in device memory 202 and executable by device processor 201. Where any component discussed herein is implemented in the form of software, any one of a number of programming languages can be employed such as, for example, C, C++, C#, Objective C, Java, Java Script, Perl, PHP, Visual Basic, Python, Ruby, Delphi, Flash, or other programming languages.

A number of software components can be stored in device memory 202 and can be executable by device processor 201. In this respect, the term “executable” can mean a program file that is in a form that can ultimately be run by device processor 201. Examples of executable programs can include a compiled program that can be translated into machine code in a format that can be loaded into a random access portion of device memory 202 and run by device processor 201, source code that can be expressed in proper format such as object code that is capable of being loaded into a random access portion of device memory 202 and executed by device processor 201, or source code that can be interpreted by another executable program to generate instructions in a random access portion of device memory 202 to be executed by device processor 201, etc. An executable program can be stored in any portion or component of device memory 202.

Device memory 202 can include both volatile and non-volatile memory and data storage components. Volatile components do not retain data values upon loss of power. Non-volatile components, on the other hand, retain data upon a loss of power. Thus, device memory 202 can comprise, for example, random access memory (RAM), read-only memory (ROM), hard disk drives, solid-state drives, USB flash drives, memory cards accessed via a memory card reader, and/or any other memory component(s) known in the art. In addition, the RAM can comprise, for example, static random-access memory (SRAM), dynamic random-access memory (DRAM), or magnetic random access memory (MRAM) and other such devices. The ROM can comprise, for example, a programmable read-only memory (PROM), an erasable programmable read-only memory (EPROM), an electrically erasable programmable read-only memory (EEPROM), or other like memory device.

Device processor 201 can represent multiple processors that operate in parallel processing circuits, respectively, or across one or more electronic mobile devices 101 and/or server(s).

Although device application 205, and other various systems described herein can be embodied in software or code executed by general purpose hardware discussed above, device application 205 can also be embodied in dedicated hardware or a combination of software/general purpose hardware and dedicated hardware. If embodied in dedicated hardware, each device application 205 can be implemented as a circuit or state machine that employs a number of technologies. These technologies can include, but are not limited to, discrete logic circuits having logic gates for implementing various logic functions upon an application of one or more data signals, application specific integrated circuits having appropriate logic gates, or other components, etc. Such technologies are generally well known by those skilled in the art and, consequently, are not described in detail herein.

The flowchart of FIG. 12 show the functionality and operation of an implementation of portions of device application 205. If embodied in software, each block can represent a module, segment, or portion of code that comprises program instructions to implement the specified logical function(s). The program instructions can be embodied in the form of source code that comprises human-readable statements written in a programming language or machine code that comprises numerical instructions recognizable by a suitable execution system such as device processor 201 in a computer system or other system. The machine code can be converted from the source code, etc. If embodied in hardware, each block can represent a circuit or a number of interconnected circuits to implement the specified logical function(s).

Although the flowchart of FIG. 12 show a specific order of execution, the order of execution can differ from what is depicted, unless otherwise stated. For example, the order of execution of two or more blocks can be rearranged relative to the order shown. Also, two or more blocks shown in succession in flowchart of FIG. 12 can be executed concurrently or with partial concurrence, unless otherwise stated. In addition, any number of counters, state variables, warning semaphores, or messages might be added to the logical flow described herein, for purposes of enhanced utility, accounting, performance measurement, or providing troubleshooting aids, etc. All such variations are within the scope of the present disclosure.

Also, any logic or application described herein that comprises software or code, including device application 205, can be embodied in any computer-readable storage medium for use by or in connection with an instruction execution system such as, device processor 201 in a computer system or other system. The logic can comprise statements including instructions and declarations that can be fetched from the computer-readable storage medium and executed by the instruction execution system.

In the context of the present disclosure, a “computer-readable storage medium” can be any non-transitory medium that can contain, store, or maintain the logic or application described herein for use by or in connection with the instruction execution system.

This above-description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure.

Various changes in the details of the illustrated operational methods are possible without departing from the scope of the following claims. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

1. An ultraviolet (UV) dryer is disclosed. The dryer can comprise a housing
a housing;
a first set of UV lights, said first set of UV lights comprising a first plurality of UV lights;
a first chamber mounted on said housing, said first chamber comprising a first access door that is configured to enclose said first set of UV lights within the inner surface of said first chamber;
a control interface placed at the front panel of said housing, said control interface comprising one or more inputs capable of operating said UV dryer.
a device memory comprising
a device application; and
a device processor that, according to the instructions from said device application
receives an input data through said one or more inputs; and
implements a set of instructions from said input data, wherein one of said set of instructions include disinfecting the daily used items using said first set of ultraviolet (UV) lights.

2. The UV dryer of claim 1 wherein said first chamber can be placed at the top portion of said housing such that said first access door configured to received daily used personal items from the top surface of said housing.

3. The UV dryer of claim 1 wherein said first chamber can be at the middle portion of said housing such that said first access door is accessible at the front surface of said housing, further wherein said first chamber configured to rotate within a central axis of said housing.

4. The UV dryer of claim 1 further comprising a second chamber positioned below said first chamber, wherein said second chamber comprises
an axle within said housing, said second chamber mounted on said axle, said second chamber configured to receive fabric materials;
a second access door, said second access door configured to enclose the fabric materials within said second chamber;
a rotary system comprising an electric motor, said rotary system configured to rotate said second chamber within said axle.

5. The UV dryer of claim 4 wherein said second chamber further comprise
a second set of UV lights comprising a second plurality of UV lights, said second set of UV lights spatially placed around the middle layer of said second chamber such that each of said second plurality of UV lights are parallel to the central axis of said second chamber; and
a slip ring assembly comprising an axle and one or more slip rings, said slip rings configured to transmit power to said second set of UV lights.

6. The UV dryer of claim 1 wherein said first access door comprises a UV filter configured to block UV rays from passing through.

7. The UV dryer of claim 4 wherein said second access door comprises a UV filter configured to block UV rays from passing through.

8. The UV dryer of claim 4 wherein said second chamber further comprise a hardware sensor, said hardware sensor placed near the front inner surface of said second chamber.

9. The UV dryer of claim 8 wherein said hardware sensor comprises a humidity sensor configured to measure moisture and air temperature within said rotating drum.

10. The UV dryer of claim 9 wherein said humidity sensor is configured to send output data to said device processor, further wherein said device processor is configured to select a preset dryer setting needed to dry the fabric materials within said rotating drum.

11. The UV dryer of claim 1 wherein said inputs of said UV dryer accessible remotely on said device application through an electronic mobile device.

12. A method for disinfecting daily used items using an ultraviolet (UV) dryer comprising the steps of
receiving on said UV dryer a set of instructions from one or more inputs, said UV dryer comprising
a first set of UV lights, said first set of UV lights comprising a first plurality of UV lights;
a first chamber comprising a first access door, said first chamber and said first access door configured to enclose said first set of UV lights within the inner surface of said first chamber;
a control interface placed at the front panel of said first chamber, said control interface comprising one or more inputs capable of manually operating said UV dryer
a device memory comprising
a device application; and
a device processor;
implementing said set of instructions received by said UV dryer, wherein one of said set of instructions include disinfecting the daily used items through said one or more sets of ultraviolet (UV) lights.

13. The method of claim 12 wherein said first chamber can be placed at the top portion of said housing such that said first access door configured to received daily used personal items from the top surface of said housing.

14. The method of claim 12 wherein said first chamber can be at the middle portion of said housing such that said first access door is accessible at the front surface of said housing, further wherein said first chamber configured to rotate within a central axis of said housing.

15. The method of claim 12 further comprising a second chamber positioned below said first chamber, wherein said second chamber comprises
an axle within said housing, said second chamber mounted on said axle, said second chamber configured to receive fabric materials;
a second access door, said second access door configured to enclose the fabric materials within said second chamber;
a rotary system comprising an electric motor, said rotary system configured to rotate said second chamber within said axle.

16. The method of claim 15 wherein said second chamber further comprise
a second set of UV lights comprising a second plurality of UV lights, said second set of UV lights spatially placed around the middle layer of said second chamber such that each of said second plurality of UV lights are parallel to the central axis of said second chamber; and
a slip ring assembly comprising an axle and one or more slip rings, said slip rings configured to transmit power to said second set of UV lights.

17. The method of claim 16 wherein said second chamber further comprise a humidity sensor configured to measure moisture and air temperature within said rotating drum.

18. The method of claim 17 wherein said humidity sensor can send output data to said device processor, further wherein said device processor selects a preset dryer setting needed to dry the fabric materials within said rotating drum.

19. The method of claim 12 wherein said inputs comprise a top-dryer start-stop button on said control interface, wherein interfacing with said top-dryer start-stop button capable of turning on said first set of UV lights of said first chamber.

20. The method of claim 12 wherein said inputs are displayed on an electronic mobile device, said inputs configured to key-in said set of instructions capable of initiating the operation of said first chamber.
